(19) United States
(12) Patent Application Publication
(10) Pub. No.: US 2024/0390051 A1
(43) Pub. Date: Nov. 28, 2024

(54) Acetabular Cup Impactor, Anteversion Guide, and Surgical Instrument

(71) Applicant: Corentec Co., Ltd., Seoul (KR)

(72) Inventors: Jung-Sung Kim, Seoul (KR); Tae-Jin Shin, Goyang-si, Gyeonggi-do (KR); A-Lyum Lim, Seoul (KR)

(21) Appl. No.: 18/688,599

(22) PCT Filed: Sep. 7, 2022
(86) PCT No.: PCT/KR2022/013450

§ 371 Date: Mar. 1, 2024

(30) Foreign Application Priority Data
Sep. 8, 2021 (KR) 10-2021-0120009

(51) Int. Cl. A61B 17/90; A61B 17/92; A61F 2/46

(52) U.S. Cl. CPC A61B 17/90; A61B 17/92; A61F 2/46

(57) ABSTRACT

An acetabular cup impactor includes: a body provided so as to apply force to an acetabular cup and having one end extending toward one side of the acetabular cup; an impactor having a coupling part formed around an axis extending from one side of the body; an alignment guide indicating an impacting direction; and an anteversion guide having a coupling mechanism coupled to the impactor at one end which extends a predetermined distance from the alignment guide, wherein the coupling mechanism of the anteversion guide is provided so as to be detachably coupled to the coupling part of the impactor, and the direction of the force applied by the coupling mechanism to the coupling portion is perpendicular to the extension direction of the impactor while a recess formed on the surface of the coupling part and the surface of the coupling part are gripped in various directions during coupling of the coupling mechanism.

### TECHNICAL FIELD

The present disclosure relates to an acetabular cup impactor, an anteversion guide, and a surgical instrument. The surgical instrument includes: the impactor including a body provided so as to apply force to an acetabular cup and having one end extending toward one side of the acetabular cup, and a coupling part formed at one side of the body around an axis along which the body extends; and the anteversion guide including an alignment guide indicating an impacting direction, and a coupling mechanism coupled to the impactor at one end extending a predetermined distance from the alignment guide, wherein the coupling mechanism of the anteversion guide is provided to be detachably coupled to the coupling part of the impactor, and the direction of the force applied by the coupling mechanism to the coupling part is perpendicular to the extension direction of the impactor while a recess formed on the surface of the coupling part and the surface of the coupling part are gripped from various directions during coupling of the coupling mechanism that is detachably coupled to the coupling part, so that the impactor and the anteversion guide are prevented from being separated from each other, and it is possible to combine and fix the impactor and the anteversion guide at a free angle for alignment, thereby increasing stability during surgery.

### BACKGROUND ART

The hip joint is a joint that connects the head of the femur and the acetabulum of the pelvis. If the hip joint is damaged for some reason and cannot perform its original function, an artificial hip joint (a prosthesis) may be surgically inserted into the hip joint area to repair damage. The prosthesis is intended to replace the function of a damaged hip joint, and typically includes an acetabular cup inserted into the acetabular region of the pelvis and assisting the movement of the femoral head, a stem that replaces the femur, and a head that replaces the femoral head. When an abnormality occurs in the hip joint, parts of the hip joint may be removed and the removed parts are replaced with artificial implants to restore the joint's motor function and reduce pain.

A conventional acetabular cup impactor is disclosed in FIG. 1. Such an acetabular cup impactor is used to install an acetabular cup in the hip joint of a patient, and after it is used to position an acetabular cup in the correct location, the cup impactor is struck with a hammer or the like at the proximal end thereof. A handle 94 is connected to an cup holder 91 adjustable by a hollow extension part 93. The extension part 93 may take a curved shape to assist a surgeon during surgery.

Meanwhile, anteversion is the angle formed by a vertical line passing through the center of the acetabulum past the anterior pelvic plane of the pelvis and the anterior and posterior margins of the acetabulum, and the additional component that aligns the anteversion when impacting the acetabular cup is called an anteversion guide. In the conventional acetabular cup impactor, the direction of the handle is guided by means of an anteversion guide 95 to impact the acetabular cup in the proper position.

The anteversion guide 95 for the conventional acetabular cup impactor 9 may be combined by using a snap hook spring method. While this method has the advantage of being able to easily combine the anteversion guide with the impactor, it has the disadvantage that the anteversion guide is easily separated from the impactor due to the force applied during impact. If the impactor and the guide are separated, the cup impactor, the anteversion guide, and even the acetabular cup inserted into the patient's body become contaminated, which is problematic. Moreover, the conventional cup impactor has a problem of making it impossible to impact the acetabular cup according to the pelvis shape that varies from patient to patient and by subdividing the insertion angle of the acetabular cup.
(Patent Document 1) U.S. Pat. No. 7,341,593 (Mar. 11, 2008)

### DISCLOSURE

### Technical Problem

The present disclosure has been made to solve the above problems, and
an objective of the present disclosure is to provide a surgical instrument including: an impactor including a body provided so as to apply force to an acetabular cup and having one end extending toward one side of the acetabular cup, and a coupling part provided at one side of the body; and an anteversion guide including an alignment guide indicating an impacting direction, and a coupling mechanism coupled to the impactor at one end extending a predetermined distance from the alignment guide, wherein the coupling mechanism of the anteversion guide is provided to be detachably coupled to the coupling part of the impactor, and the direction of the force applied by the coupling mechanism to the coupling part forms a predetermined angle with the extension direction of the impactor, preventing separation of the impactor and the anteversion guide.

Another objective of the present disclosure is to provide the surgical instrument in which the coupling part includes a plurality of recesses formed along at least a portion of the circumference of the impactor and having a constant central angle from the center, and the coupling mechanism that is detachably coupled to the coupling part includes: a coupling frame provided to surround at least a portion of the coupling part; and a grip that grips the coupling part by applying force from one side of the coupling part, wherein one end of the grip protrudes inward and is fixed to the recesses, enabling stable coupling of the impactor and the anteversion guide.

Still another objective of the present disclosure is to provide the surgical instrument in which the coupling frame is hinge coupled with the grip, and an elastic member is provided between the other side of the grip and the coupling frame, so that the grip rotates depending on the force applied to the grip and engages or disengages from the coupling part, allowing a surgeon to easily combine the impactor and the anteversion guide.

Still another objective of the present disclosure is to provide the surgical instrument in which the grip includes: a pressing part that is connected to the elastic member and transmits the force applied to the grip to the elastic member; and a gripping part that grips the coupling part according to the rotation of the grip, wherein the gripping part includes a first portion bent and extended from one end thereof, and a second portion whose inner surface extends straight between the first portion and the part where the grip is hinge coupled, and when the coupling mechanism is coupled, the second portion comes into contact with the surface of the coupling part and applies force to grip the coupling part, so that the impactor and the anteversion guide are stably coupled.

Still another objective of the present disclosure is to provide surgical instrument in which the coupling frame includes legs provided to surround at least a portion of the coupling part, and when the coupling mechanism is coupled, the first portion is accommodated in the recesses of the coupling part, and the second portion protrudes from the inner surfaced of the legs and comes into contact with the surface of the coupling part, thereby providing stable force.

Still another objective of the present disclosure is to provide the surgical instrument in which the coupling frame includes a stop rod that extends at a predetermined distance from the portion where the grip is hinge coupled, and the grip further includes a stopper provided to surround at least a portion of the stop rod to limit the rotation of the grip, preventing excessive force from being applied to the coupling part or the grip from falling off.

Still another objective of the present disclosure is to provide the surgical instrument in which two grips are provided centered on the coupling part to hold the coupling part on both sides of the coupling part.

Still another objective of the present disclosure is to provide the surgical instrument in which the direction of the force applied by the coupling mechanism to the coupling part is perpendicular to the extension direction of the impactor, so that the force applied to the impactor does not affect the coupling with the anteversion guide.

Still another objective of the present disclosure is to provide the surgical instrument in which the body of the impactor extends forming a hollow, and the impactor further includes an anchor at least partially extending through the interior of the body and having one end capable of engaging with the acetabular cup, wherein the anchor includes: a shaft extending within the body; a tip end portion coupled to the acetabular cup at one end of the shaft; and a knob for rotating the shaft and the tip end portion, enabling easy installation of the acetabular cup.

Still another objective of the present disclosure is to provide the surgical instrument that is hygienic and convenient to clean since a plurality of through holes are formed along the circumference of the body.

### Technical Solution

The present disclosure is implemented by an embodiment having the following configuration to achieve the above mentioned objectives.

According to an embodiment of the present disclosure, there is provided a surgical instrument including: an impactor including a body provided to apply force to an acetabular cup and having an end thereof extending toward a first side of the acetabular cup, and a coupling part provided at a side of the body; and an anteversion guide including an alignment guide indicating an impacting direction, and a coupling mechanism coupled to the impactor at an end thereof extending a predetermined distance from the alignment guide, wherein the coupling mechanism of the anteversion guide may be provided to be detachably coupled to the coupling part of the impactor, and a direction of force applied by the coupling mechanism to the coupling part may form a predetermined angle with an extension direction of the impactor.

According to another embodiment of the present disclosure, the coupling part may include a plurality of recesses formed along at least a portion of a circumference of the impactor while having a constant central angle from a center, and the coupling mechanism that is detachably coupled to the coupling part may include: a coupling frame provided to surround at least a portion of the coupling part; and a grip that grips the coupling part by applying force from a side of the coupling part, wherein an end of the grip may protrude inward and may be fixed to one of the recesses.

According to still another embodiment of the present disclosure, the coupling frame may be hinge coupled with the grip, and an elastic member may be provided between a second side of the grip and the coupling frame, so that the grip may rotate by force applied thereto to engage with or disengage from the coupling part.

According to still another embodiment of the present disclosure, the grip may include: a pressing part that is connected to the elastic member and transmits the force applied to the grip to the elastic member; and a gripping part that grips the coupling part according to rotation of the grip, wherein the gripping part may include: a first portion bent and extended inward from an end thereof; and a second portion whose inner surface extends straight between the first portion and a part where the grip is hinge coupled, wherein when the coupling mechanism is coupled, the second portion may contact a surface of the coupling part.

According to still another embodiment of the present disclosure, the coupling frame may include a leg provided to surround at least a portion of the coupling part, and when the coupling mechanism is coupled, the first portion may be received in one of the recesses of the coupling part and the second portion may protrude from an inner surface of the leg and contact a surface of the coupling part.

According to still another embodiment of the present disclosure, the coupling part may further include a protrusion formed alternately with each of the recesses from a surface of the coupling part, and the second portion may be formed with an inner surface thereof recessed to accommodate the protrusion.

According to still another embodiment of the present disclosure, the coupling frame may include a stop rod that extends a predetermined distance from a part where the grip is hinge coupled, and the grip may further include a stopper provided to surround at least a portion of the stop rod to limit the rotation of the grip.

According to still another embodiment of the present disclosure, two grips may be provided centered on the coupling part to hold the coupling part on opposite sides of the coupling part.

According to still another embodiment of the present disclosure, a direction of force applied by the coupling mechanism to the coupling part is perpendicular to an extension direction of the impactor.

According to still another embodiment of the present disclosure, the body of the impactor may extend forming a hollow, and the impactor may further include an anchor at least partially extending through an interior of the body and having an end capable of engaging with the acetabular cup.

According to still another embodiment of the present disclosure, the anchor may include: a shaft extending within the body; a tip end portion coupled to the acetabular cup at an end of the shaft; and a knob for rotating the shaft and the tip end portion.

According to still another embodiment of the present disclosure, a plurality of through holes are formed along a circumference of the body.

### Advantageous Effects

The present disclosure can achieve the following effects by the above-mentioned embodiments and the configuration, combination, and use relationship described below.

According to the present disclosure, it is possible to prevent separation of an impactor and an anteversion guide since a surgical instrument of the present disclosure includes: the impactor including a body provided so as to apply force to an acetabular cup and having one end extending toward one side of the acetabular cup, and a coupling part provided at one side of the body; and the anteversion guide including an alignment guide indicating an impacting direction, and a coupling mechanism coupled to the impactor at one end extending a predetermined distance from the alignment guide, wherein the coupling mechanism of the anteversion guide is provided to be detachably coupled to the coupling part of the impactor, and the direction of the force applied by the coupling mechanism to the coupling part forms a predetermined angle with the extension direction of the impactor.

According to the present disclosure, stable coupling of an impactor and an anteversion guide is possible since the coupling part includes a plurality of recesses formed along at least a portion of the circumference of the impactor and having a constant central angle from the center, and the coupling mechanism that is detachably coupled to the coupling part includes: a coupling frame provided to surround at least a portion of the coupling part; and a grip that grips the coupling part by applying force from one side of the coupling part, wherein one end of the grip protrudes inward and is fixed to the recesses.

According to the present disclosure, it is possible for a surgeon to combine an impactor and an anteversion guide in a simple way since the coupling frame is hinge coupled with the grip, and an elastic member is provided between the other side of the grip and the coupling frame, so that the grip rotates depending on the force applied to the grip and engages or disengages from the coupling part.

According to the present disclosure, it is possible to provide a surgical instrument that stably combines an impactor and an antenna guide since the grip includes: a pressing part that is connected to the elastic member and transmits the force applied to the grip to the elastic member; and a gripping part that grips the coupling part according to the rotation of the grip, wherein the gripping part includes a first portion bent and extended from one end thereof, and a second portion whose inner surface extends straight between the first portion and the part where the grip is hinge coupled, and when the coupling mechanism is coupled, the second portion comes into contact with the surface of the coupling part and applies force to grip the coupling part.

According to the present disclosure, it is possible to provide stable force since the coupling frame includes legs provided to surround at least a portion of the coupling part, and when the coupling mechanism is coupled, the first portion is accommodated in the recesses of the coupling part, and the second portion protrudes from the inner surfaced of the legs and comes into contact with the surface of the coupling part.

According to the present disclosure, it is possible to prevent excessive force from being applied to the coupling part or the grip from falling off since the coupling frame includes a stop rod that extends at a predetermined distance from the portion where the grip is hinge coupled, and the grip further includes a stopper provided to surround at least a portion of the stop rod to limit rotation of the grip.

According to the present disclosure, it is possible to provide a surgical instrument in which two grips are provided centered on the coupling part to hold the coupling part on both sides of the coupling part.

According to the present disclosure, it is possible to ensure that the force applied to an impactor does not affect the combination with an antenna guide since the direction of the force applied by the coupling mechanism to the coupling part is perpendicular to the extension direction of the impactor.

According to the present disclosure, it is possible to provide a surgical instrument that allows easy acetabular cup installation since the body of the impactor extends forming a hollow, and the impactor further includes an anchor at least partially extending through the interior of the body and having one end capable of engaging with the acetabular cup, wherein the anchor includes: a shaft extending within the body; a tip end portion coupled to the acetabular cup at one end of the shaft; and a knob for rotating the shaft and the tip end portion.

According to the present disclosure, it is possible to provide a surgical instrument that is hygienic and convenient to clean since a plurality of through holes are formed along the circumference of the body.

### DESCRIPTION OF DRAWINGS

FIG. 1 is a side view of a conventional acetabular cup impactor;

FIG. 2 is a perspective view of a surgical instrument 1 according to a preferred embodiment of the present disclosure;

FIG. 3 is an exploded perspective view of the surgical instrument 1 according to the preferred embodiment of the present disclosure;

FIG. 4 is an enlarged view of a portion of the surgical instrument 1 according to the preferred embodiment of the present disclosure;

FIG. 5 is a view showing a coupling part 13 and an anteversion guide 30 according to an embodiment of the present disclosure being coupled;

FIG. 6 is an exploded perspective view of the anteversion guide 30 according to the embodiment of the present disclosure;

FIG. 7 is a cross-sectional view showing the combination of a grip 333 and the coupling part 13 according to the embodiment of the present disclosure; and

FIG. 8 is a cross-sectional view showing the combination of a grip 333 and a coupling part 13 according to another embodiment of the present disclosure.

### BEST MODE

Hereinafter, an acetabular cup impactor, an anteversion guide, and a surgical instrument according to the present disclosure will be described in detail with reference to the attached drawings. It should be noted that like components in the drawings are represented by like numerals wherever possible. In addition, detailed descriptions of well-known functions and configurations that may unnecessarily obscure the gist of the present disclosure are omitted. Unless otherwise specified, all terms in this specification have the same general meaning as understood by a person skilled in the art to which the present disclosure pertains, and if the general meaning conflicts with the meaning of a term used in this specification, the definition used in this specification shall apply.

Throughout the specification, when a part is said to “include” a certain component, this does not mean excluding other components unless specifically stated to the contrary, but means that other components may also be included, and terms such as “˜unit (part)” used in the specification refer to a unit (part) that processes at least one function or operation. In addition, when it is said that certain components are “connected”, this is not limited to the components being connected by direct contact with each other, but also includes being connected through other components, and may mean that the components are arranged to transmit a certain amount of force or energy even if they are not connected. Terms such as “1st˜” and “2nd˜” may be used to indicate the same or substantially the same configuration in a different order, and may be interpreted as substantially the same configuration as those without “1st”, “2nd”, etc. Hereinafter, the present disclosure will be described in detail by explaining preferred embodiments of the present disclosure with reference to the accompanying drawings.

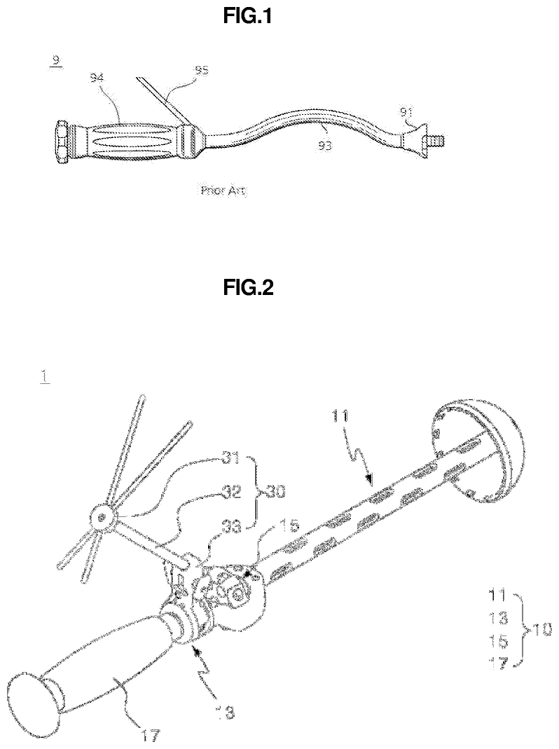

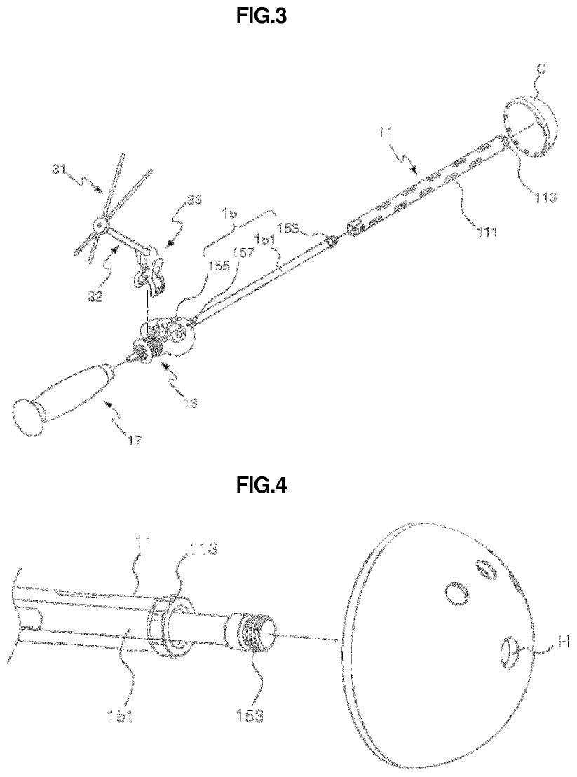

FIG. 2 is a perspective view of a surgical instrument 1 having a combined structure of an acetabular cup impactor and an anteversion guide according to a preferred embodiment of the present disclosure, and FIG. 3 is an exploded perspective view of the surgical instrument 1 having a combined structure of an acetabular cup impactor and an anteversion guide according to the preferred embodiment of the present disclosure. The surgical instrument according to the present disclosure is characterized in that, when combining an impactor and an anteversion guide, the direction in which the force is applied when the anteversion guide grips the impactor is perpendicular to the direction in which the force is applied during impacting, so that the force applied when impacting the acetabular cup is not transmitted to the coupling structure formed between the impactor and the anteversion guide, thereby preventing separation of the impactor and the anteversion guide, the impactor and the anteversion guide may be combined at any angle since the angle to align the anteversion guide is subdivided, and the coupling stability is increased since the anteversion guide grips the impactor at several points. The surgical instrument 1 includes an impactor 10 and an anteversion guide 30.

The impactor 10 is provided to apply force to an acetabular cup and is used to seat an acetabular cup on the patient's acetabulum. The impactor 10 is provided so that one end thereof is in contact with or connected to the concave side of an acetabular cup, and is provided to enable impacting by force applied by hitting, etc. at the other end thereof. When inserting an acetabular cup, the insertion angle is important. Thus, the impactor 10 is connected to an acetabular cup C and then combined with the anteversion guide 30, which will be described later, so that the insertion and impacting direction may be guided by the anteversion guide 30. The impactor 10 may include a body 11, a coupling part 13, an anchor 15, and a handle 17.

The body 11 has one end extending toward the concave side of the acetabular cup and may be formed to transmit an external force applied through the other end of the impactor. In the embodiment of the present disclosure, the body 11 may extend straight along the axis along which the impactor 10 extends, but the body 11 being extended with a curvature as in the conventional case shown in FIG. 1 is not excluded from the scope of the present disclosure.

In the embodiment of the present disclosure, the body 11 is provided in a hollow tubular shape and may accommodate the anchor 15 therein for fastening to the acetabular cup C. Accordingly, one end of the anchor 15, which will be described later, is connected to the acetabular cup C, and the body 11 may act to apply force to the acetabular cup side. However, in another embodiment of the present disclosure not shown, the body 11 may be extended without a hollow, and one end of the body may be connected to the acetabular cup C by a predetermined fastening method.

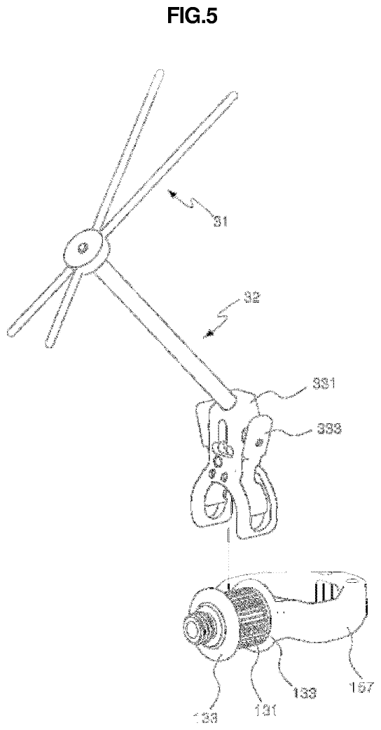

The body 11 may include a plurality of through holes 111 formed along the circumference of the body, and a transmission part 113 that transmits external force transmitted through the body at one end thereof on the acetabular cup side. The plurality of through holes 111 are formed along the circumference of the body 11 while penetrating the body, thereby allowing liquid or air to pass through the body 11 inside and outside, thereby enabling easy cleaning. Referring to a preferred embodiment of the present disclosure shown in FIG. 4, the transmission part 113 may have a cross-sectional thickness that is greater than the thickness of the body 11 by a predetermined amount in order to efficiently transmit the external force transmitted through the body 11 to the acetabular cup, and may also have a non-circular cross-section. As the transmission part 113 applies force in the axial direction in which the impactor extends around a hole H of the acetabular cup, the acetabular cup C may be implanted in the direction and position desired by a surgeon within the patient's pelvis.

Referring to FIGS. 2, 3, and 5, the coupling part 13 is a portion provided along the circumference of the impactor 10 on one side of the body 11, and may guide the insertion direction of the acetabular cup by combining with the anteversion guide 30, which will be described later. The coupling part 13 is provided to a predetermined length in a portion of the impactor 10 and may include a plurality of recesses 131 and protruding jaws 133.

The recess 131 is a groove formed on the surface of the coupling part, and the recesses 131 may be formed along the circumference of the impactor with a constant central angle from the center. The recesses 131 may be formed in the longitudinal direction in which the impactor extends. The center of the impactor from one recess 131 to the other recess 131, that is, the central angle from the axis along which the impactor extends, may be a certain angle such as 10 degrees, 15 degrees, 30 degrees, etc. However, the recesses may be formed in some parts with a relatively small central angle of around 5 degrees, and in other parts with a relatively large central angle of around 10 degrees. Although the recesses 131 may be formed on a portion of the circumference of the impactor and/or the coupling part with a central angle of 90 degrees, 120 degrees, 150 degrees, 180 degrees, etc., at which all of the recesses are formed, it is preferable that the recesses 131 is formed along the entire circumference of the impactor and/or the coupling part. The coupling of the recesses 131 and the coupling mechanism described later will be described later.

The protruding jaw 133 is formed to protrude outward from the surface of the coupling part at each end of the plurality of recesses 131. The protruding jaws 133 may be provided to ensure that the coupling part 13 and the anteversion guide 30 are connected and are stably fastened without shaking upon impacting. The length between the two protruding jaws 133 may correspond to or be equal to the anteroposterior length of the coupling mechanism and/or coupling frame connected to the coupling part 13, which will be described later. In the preferred embodiment of the present disclosure, the scope of the present disclosure does not exclude that a part of the protruding jaw 133 is formed integrally with another component of the impactor, for example, a coverage 157, which will be described later.

Referring again to FIGS. 2 to 4, the anchor 15 is configured so that one end thereof may be coupled to the acetabular cup. The anchor 15 is provided so that at least a portion thereof extends through the inside of the body 11 and engages with the hole H of the acetabular cup C. The anchor 15 may include a shaft 151, a tip end portion 153, a knob 155, and the coverage 157.

The shaft 151 is a part that extends within the body 11 as described above, and may have the tip end portion 153 at one end thereof, which is fastened to the hole H of the acetabular cup. The tip end portion 153 has a shape suitable for fastening with the hole H of the acetabular cup C. Although in the embodiment of the present disclosure, the shaft 151 and the tip end portion 153 may be rotated by the knob 155 and screwed into the hole H, the shaft 151 and the tip end portion 153 may be formed to enable various fastening types such as tapered coupling and interference fit coupling depending on the hole H of the acetabular cup C. The knob 155 is configured to allow the shaft 151 and the tip end portion 153 to rotate on the other side of the shaft, and the knob 155 is preferably exposed to the outside of the body 11 so that a surgeon may hold and turn the knob 155. The coverage 157 is provided to surround the knob 155, and may be formed substantially integrally with the body 11 or may be fastened to the body 11 to transmit the force applied to the impactor 10 through the body 11 to the acetabular cup.

The handle 17 is provided at one end of the impactor 10 so that a surgeon may hold the impactor, and has a substantially flat surface at the end, so that force may be applied to the acetabular cup by hitting one end of the handle.

The anteversion guide 30 is a component that functions as a direction indicator to ensure that the acetabular cup is positioned in the correct direction when installing the acetabular cup. When impacting with the anteversion guide 30 aligned, if the anteversion guide 30 and the impactor 10 are separated by external force, it is difficult to know whether a surgeon is impacting in the correct direction. The anteversion guide 30 of the present disclosure may include an alignment guide 31 indicating impacting direction, a column 32 extending a predetermined length from the alignment guide 31, and a coupling mechanism 33 detachably coupled to the coupling part 13 to provide a stable coupling.

The alignment guide 31 preferably has a shape in which rods extending in two directions intersect. A surgeon may rotate or move the anteversion guide 30 to place the alignment guide 31 in an appropriate position and direction. The column 32 extends from the alignment guide 31 to the coupling mechanism 33.

The coupling mechanism 33 may be formed at one end of the column 32 extending a predetermined distance from the alignment guide and is provided to be detachably coupled to the coupling part 13 of the impactor 10. In the embodiment of the present disclosure, when the coupling mechanism 33 is coupled, the direction of the force applied by the coupling mechanism 33 to the coupling part 13 forms a predetermined angle with, preferably perpendicular to, the extension direction of the impactor, that is, the direction in which the force is transmitted during impact, so that even during impact, the coupling mechanism 33 may not be separated from the impactor 10. The coupling mechanism 33 includes a coupling frame 331, an elastic member 332 and a grip 333.

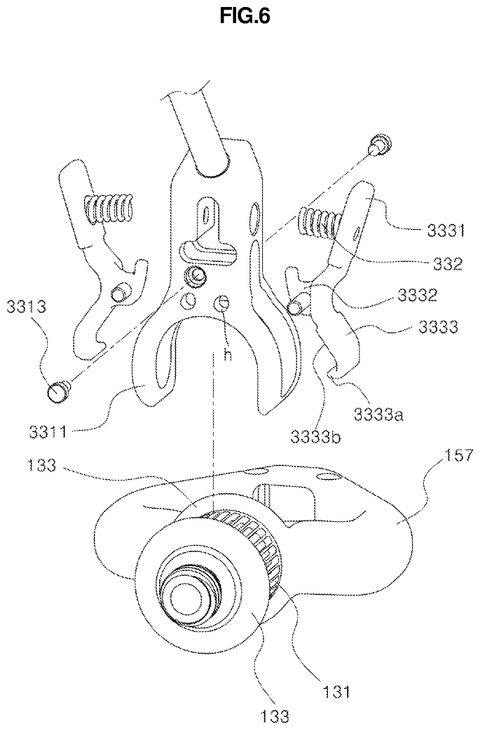

Referring to FIGS. 5 to 7, the coupling frame 331 may be provided to surround at least a portion of the coupling part 13. The elastic member 332 and the grip 333, which will be described later, are connected to the coupling frame 331 to grip the recesses 131 of the coupling part 13. Viewed as a whole, the coupling frame 331 has a shape in which a portion thereof extends from the column 32 and the other portion thereof surrounds the coupling part 13 having a circular perimeter, but has a cavity formed on the side thereof so that the grip 333, which will be described later, may move forward or backward into the recess 131 of the coupling part 13 by means of the cavity. The coupling frame 331 is hinge coupled to the grip 333 at a predetermined portion so that the grip 333 may rotate around a hinge coupled portion h.

The coupling frame 331 may include a leg 3311 formed to surround at least part of the coupling part, and a stop rod 3313 that limits the turning radius of the grip 333. The leg 3311 has a U-shape so that the coupling mechanism 33 may easily approach the coupling part 13 from one side of the impactor, but has a cavity in the portion corresponding to the area where the recesses 131 are formed, so that when the grip 333 rotates, one end of the grip 333 may be fixed to the recesses 131 through the empty space of the cavity. In addition, the stop rod 3313 may be provided to extend across the coupling frame 331 at a predetermined distance from the portion h where the grip is hinge coupled. The stop rod 3313 may limit the rotation radius of the grip 333 as a stopper 3332 of the grip 333, which will be described later, is provided from the grip 333 in a shape that partially surrounds the stop rod 3313.

The elastic member 332 may be provided to extend from one side of the coupling frame and be connected to the grip, and may transmit the force pressing the grip 333 and restore the state of the grip 333 by elastic force.

The grip 333 is provided to grip the coupling part 13, which is the subject to be combined, by applying force from one side of the coupling part. Preferably, one end of the grip 333 is protruded and/or bent and extended inward and is fixed to the recesses 131 to enable stable coupling or gripping. The grip 333 is hinge coupled with the coupling frame 331 and rotates according to the force applied to the grip to engage or disengage from the coupling part. In the preferred embodiment of the present disclosure, two grips 333 may be respectively provided on opposite sides of the coupling frame 331, and the grip 333 includes a pressing part 3331, the stopper 3332, and a gripping part 3333.

The pressing part 3331 is connected to the elastic member on one side of the grip 333 and is provided to transmit the force applied to the grip to the elastic member 332. The pressing part 3331 may be a push button provided with a predetermined width and length. A surgeon may manipulate the pressing part 3331 to cause one end of the gripping part 3333 to advance into the recesses 131 and be fixed therein or to retract outward from the recesses 131. The pressing part 3331 is preferably provided in the opposite direction to the gripping part 3333 based on the portion where the grip 333 is hinge coupled to the coupling frame 331.

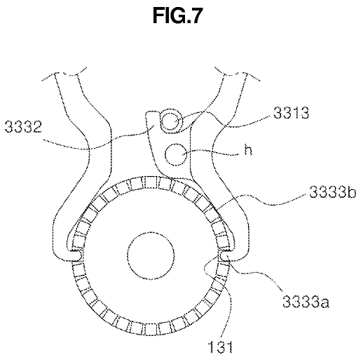

The stopper 3332 has a shape that surrounds at least a portion of the stop rod 3313 described above near the portion where the grip is hinge coupled, and is a part that protrudes from the grip 333. The stopper 3332 comes into contact with the stop rod 3313 when the grip 333 is coupled with the recesses 131, as shown in FIG. 7, and limits the rotation of the grip 333. Furthermore, the stopper 3332 may prevent one end of the grip from damaging the recesses 131. The stopper 3332 may have an inner end that preferably protrudes toward the pressing part so that the stop rod is caught thereinside.

The gripping part 3333 is a part that substantially forms a connection with the coupling part 13. The gripping part 3333 may contact the surface of the coupling part 13 according to the rotation of the grip to grip the coupling part, or may advance and be fixed into the recesses 131 formed in the coupling part. The gripping part 3333 may include a first portion 3333a bent and extended inward from one end thereof, and a second portion 3333b extending substantially straight between the first portion and the part where the grip is hinge coupled. Because the inner surface of the second portion 3333b extends substantially in a straight line, when the gripping part 3333 is combined with the coupling part within the cavity of the U-shaped leg 3311, the second portion 3333b may protrude from the inner surface of the leg inward, that is, toward the coupling part, and come into contact with the surface of the coupling part.

Hereinafter, the coupling of the coupling mechanism 33 and the coupling part 13 will be described. In the initial preparation state where the pressing part 3331 is not pressed, the first portion 3333a protrudes inside the leg 3311 without the grip 333 rotating since no force acts on the elastic member 332. When a surgeon presses the pressing part 3331 to combine the anteversion guide 30 and the impactor 10, the grip 333 rotates around the portion hinge coupled to the coupling frame 331. In this state, the surgeon may make the coupling mechanism 33 approach the coupling part from any free direction to achieve alignment. When the force acting on the pressing part 3331 is removed, the gripping part 3333 advances toward the coupling part due to elastic force, and the first portion 3333a of one end of the gripping part is received and fixed in the recesses 131, and the second portion 3333b comes into contact with the surface of the coupling part. The first portion 3333a is received and fixed in the recess 131, while the second portion 3333b strongly grips the surface of the coupling part 13 due to elastic force. Thus, force is applied from various directions around the impactor 10 to strongly couple the anteversion guide 30 and the impactor 10, and all forces acting on the coupling part 13 are perpendicular to the axis along which the impactor 10 extends, so that the possibility of separation of the anteversion guide 30 and the impactor 10 due to the force applied during impact may be dramatically reduced. In addition, unlike conventional surgical instruments, which may only be assembled at a specific angle and need to be manipulated to adjust the angle after assembly, in the present disclosure, the coupling mechanism 33 and the coupling part 13 may be coupled while the pressing part is pressed in the direction of the coupling frame, so that the coupling mechanism 33 and the coupling part 13 may be freely attached and detached at a desired angle. Since the coupling mechanism 33 and the coupling part 13 may be combined at any angle during a surgical process, the process of adjusting the angle for alignment after combining the anteversion guide 30 and the impactor 10 may be omitted.

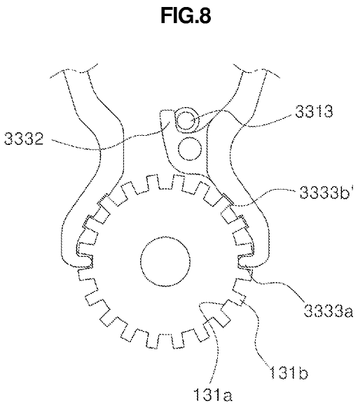

Referring to another embodiment of the present disclosure shown in FIG. 8, the coupling part 13 may include a recess 131a recessed from the surface, and a protrusion 131b formed alternately with the recess 131a. In this case, the coupling part may have a shape like a gear wheel. In addition, the inner surface of the second portion 3333b may be recessed to accommodate the protrusion 131b. When a surgeon combines the anteversion guide 30 and the impactor 10 and removes the force applied to the pressing part 3331, a more stable coupling may be provided by the protrusion 131b being accommodated in the recessed portion of the inner surface of the second portion 3333b.

The above detailed description is illustrative of the present disclosure. In addition, the foregoing describes preferred embodiments of the present disclosure, and the present disclosure may be used in various other combinations, modifications, and environments. That is, the present disclosure may be changed or modified within the scope of the inventive concept disclosed herein, the scope equivalent to the written disclosure, and/or the skill or knowledge in the art. The written embodiments illustrate the best state for implementing the technical idea of the present disclosure, and various changes required for specific application fields and uses of the present disclosure are also possible. Therefore, the above detailed description of the invention is not intended to limit the invention to the disclosed embodiments. Furthermore, the appended claims should be construed to include other embodiments as well.

1. A surgical instrument, comprising:
an impactor comprising:
a body provided to apply force to an acetabular cup and having an end thereof extending toward a first side of the acetabular cup; and
a coupling part provided at a side of the body; and
an anteversion guide comprising:
an alignment guide indicating an impacting direction; and
a coupling mechanism coupled to the impactor at an end thereof extending a predetermined distance from the alignment guide,
wherein the coupling mechanism of the anteversion guide is provided to be detachably coupled to the coupling part of the impactor, and a direction of force applied by the coupling mechanism to the coupling part forms a predetermined angle with an extension direction of the impactor.

2. The surgical instrument of claim 1, wherein the coupling part comprises a plurality of recesses formed along at least a portion of a circumference of the impactor while having a constant central angle from a center, and
the coupling mechanism that is detachably coupled to the coupling part comprises:
a coupling frame provided to surround at least a portion of the coupling part; and
a grip that grips the coupling part by applying force from a side of the coupling part,
wherein an end of the grip protrudes inward and is fixed to one of the recesses.

3. The surgical instrument of claim 2, wherein the coupling frame is hinge coupled with the grip,
an elastic member is provided between a second side of the grip and the coupling frame, so that the grip rotates by force applied thereto to engage with or disengage from the coupling part, and
the grip comprises:
a pressing part that is connected to the elastic member and transmits the force applied to the grip to the elastic member; and
a gripping part that grips the coupling part according to rotation of the grip.

4. The surgical instrument of claim 3, wherein the coupling frame comprises a leg provided to surround at least a portion of the coupling part, and
the gripping part comprises:
a first portion bent and extended inward from an end thereof; and
a second portion whose inner surface extends straight between the first portion and a part where the grip is hinge coupled,
wherein when the coupling mechanism is coupled, the first portion is received in one of the recesses of the coupling part and the second portion protrudes from an inner surface of the leg and contacts a surface of the coupling part.

5. The surgical instrument of claim 3, wherein the coupling frame comprises a stop rod that extends a predetermined distance from a part where the grip is hinge coupled, and the grip further comprises a stopper provided to surround at least a portion of the stop rod to limit the rotation of the grip.

6. The surgical instrument of claim 1, wherein the body of the impactor extends forming a hollow, and the impactor further comprises an anchor at least partially extending through an interior of the body and having an end capable of engaging with the acetabular cup,
wherein the anchor comprises:
a shaft extending within the body;
a tip end portion coupled to the acetabular cup at an end of the shaft; and
a knob for rotating the shaft and the tip end portion.

7. The surgical instrument of claim 6, wherein a plurality of through holes are formed along a circumference of the body.

8. An impactor, comprising:
a body provided to apply force to an acetabular cup and having an end thereof extending toward a first side of the acetabular cup; and
a coupling part provided at a side of the body,
wherein the body extends forming a hollow, and the impactor further comprises an anchor at least partially extending through an interior of the body and having an end capable of engaging with the acetabular cup,
wherein the anchor comprises:
a shaft extending within the body; and
a tip end portion coupled to the acetabular cup at an end of the shaft, and the coupling part comprises:
a plurality of recesses formed along at least a portion of a circumference of the impactor in a longitudinal direction in which the impactor extends while having a constant central angle from a center; and
a protruding jaw that protrudes outward from a surface of the coupling part on at least one end of the plurality of recesses.

9. An anteversion guide, comprising:
an alignment guide indicating an impacting direction; and
a coupling mechanism provided at an end of the anteversion guide extending a predetermined distance from the alignment guide,
wherein the coupling mechanism comprises:
a coupling frame with at least part thereof having a U-shape; and
a grip that grips a subject to be combined,
wherein an end of the grip protrudes inward.

10. The anteversion guide of claim 9, wherein the coupling frame is hinge coupled with the grip,
an elastic member is provided between a second side of the grip and the coupling frame, so that the grip rotates by force applied thereto to engage with or disengage from the coupling part, and
the grip comprises:
a pressing part that is connected to the elastic member and transmits the force applied to the grip to the elastic member; and
a gripping part that grips the subject to be combined as the grip rotates.

11. The anteversion guide of claim 10, wherein the gripping part comprises:
a first portion bent and extended inward from an end thereof; and
a second portion whose inner surface extends straight between the first portion and a part where the grip is hinge coupled,
wherein when the coupling mechanism is coupled, the second portion protrudes from an inner surface of the coupling frame.

12. The surgical instrument of claim 2, wherein the body of the impactor extends forming a hollow, and the impactor further comprises an anchor at least partially extending through an interior of the body and having an end capable of engaging with the acetabular cup,
wherein the anchor comprises:
a shaft extending within the body;
a tip end portion coupled to the acetabular cup at an end of the shaft; and
a knob for rotating the shaft and the tip end portion.

13. The surgical instrument of claim 3, wherein the body of the impactor extends forming a hollow, and the impactor further comprises an anchor at least partially extending through an interior of the body and having an end capable of engaging with the acetabular cup,
wherein the anchor comprises:
a shaft extending within the body;
a tip end portion coupled to the acetabular cup at an end of the shaft; and
a knob for rotating the shaft and the tip end portion.

14. The surgical instrument of claim 4, wherein the body of the impactor extends forming a hollow, and the impactor further comprises an anchor at least partially extending through an interior of the body and having an end capable of engaging with the acetabular cup,
wherein the anchor comprises:
a shaft extending within the body;
a tip end portion coupled to the acetabular cup at an end of the shaft; and
a knob for rotating the shaft and the tip end portion.

15. The surgical instrument of claim 5, wherein the body of the impactor extends forming a hollow, and the impactor further comprises an anchor at least partially extending through an interior of the body and having an end capable of engaging with the acetabular cup,
wherein the anchor comprises:
a shaft extending within the body;
a tip end portion coupled to the acetabular cup at an end of the shaft; and
a knob for rotating the shaft and the tip end portion.
